(19) United States
(12) Patent Application Publication
(10) Pub. No.: US 2010/0174550 A1
(43) Pub. Date: Jul. 8, 2010

(54) METHOD AND SYSTEM FOR ONLINE INVENTORY AND COST MANAGEMENT

(75) Inventors: Jennifer Reverendo, Marysville, OH (US); Donna Kolar, Gurnee, IL (US); Richard Landrove Rea, JR., Rolling Meadows, IL (US); William Frank Miller, Mundelein, IL (US)

Correspondence Address: ARENT FOX LLP, 1050 CONNECTICUT AVENUE, N.W., SUITE 400, WASHINGTON, DC, 20036, US

(73) Assignee: Allegiance Corporation, McGaw Park, IL (US)

(21) Appl. No.: 12/496,131

(22) Filed: Jul. 1, 2009

Related U.S. Application Data
(60) Provisional application No. 61/129,531, filed on Jul. 2, 2008.

(51) Int. Cl. G06Q 10/00; G06Q 50/00; G06F 17/30

(52) U.S. Cl. 705/2; 705/28; 705/10; 705/348; 707/754; 707/E17.059; 707/E17.014; 707/758

(57) ABSTRACT

The present invention relates to a method and system for procedure pack inventory management. The system provides access to data relating to healthcare facilities, custom and standard procedure pack data, component data for each procedure pack, and vendor data. The system may filter the access data and output the results of the filterings on a display.

### RELATED APPLICATIONS

This application claims priority to U.S. Provisional Application No. 61/129,531, titled “Method and System for Online Inventory and Cost Management,” filed Jul. 2, 2008, which is incorporated by reference herein in its entirety. This application also is related to U.S. application Ser. No. 11/027,504 titled SYSTEM AND METHOD FOR MANAGING PROCEDURE PACKS COSTS filed Dec. 30, 2004, which is a continuation-in-part of U.S. application Ser. No. 10/098,978 titled SYSTEM AND METHOD FOR MANAGING PROCEDURE PACKS filed Mar. 15, 2002. The entirety of each of these related applications is incorporated by reference herein.

### BACKGROUND OF THE INVENTION

1. Field of the Invention

Aspects of the present invention relate to methods and systems for providing decision support in creating, monitoring and/or modifying inventory, viewing inventory information, including component details and/or images, and monitoring inventory spending and cost saving opportunities. Specifically, aspects of the present invention relate to methods and systems for providing online decision support relating to procedure pack inventory and cost management in a hospital or other healthcare provider setting.

2. Background of the Technology

Many healthcare providers today use standard and/or custom procedure packs (i.e., packages or kits containing disposable items intended for use in specific medical procedures) in order to provide healthcare services to patients. In addition, there are automated systems that provide information regarding indirect costs that are incurred due to a specific healthcare provider's order management practices. For example, a provider's overall costs for procedure packs may be higher than necessary because the provider orders more packs than necessary, because different locations within the same healthcare organization use different packs for similar procedures, or because the user who is creating or ordering a pack is unaware of lower cost alternatives for components that are contained in other standard and/or custom packs.

Nevertheless, there is currently no method and system that allows access to data relating to multiple participating healthcare providers, the custom and/or standard procedure packs (also interchangeably referred to herein as “kits”) used by the multiple participating healthcare providers, a list of all components within each kit, and the vendors providing each component within a kit. In addition, there is currently no method and system that provides the capability to search, filter and compare the accessed data for each and/or multiples of: (a) one or more specific participating healthcare providers; (b) one or more custom and/or standard procedure packs used by the multiple participating healthcare providers; (c) one or more of the components within each kit; (d) one or more of the vendors providing each component within a kit; and (e) keyword and/or other identifier that designates a specific component within a kit. There is a further need in the art for a method and system that provides the capability to search, filter and compare accessed data for all of the participating hospitals using multiple criteria.

### SUMMARY OF THE INVENTION

Aspects of the present invention solve the above problems, and others, by providing systems and methods for accessing data relating to multiple participating healthcare providers, the custom and/or standard procedure packs used by the multiple participating healthcare providers, a list of all components within each kit, and the vendors providing each component within a kit. Further, aspects of the present invention provide a system and method for searching, filtering and comparing the accessed data for each and/or multiples of: (a) one or more specific participating healthcare providers; (b) one or more custom and/or standard procedure packs used by the multiple participating healthcare providers; (c) one or more of the components within each kit; (d) one or more of the vendors providing each component within a kit; and (e) keyword and/or other identifier that designates a specific component within a kit. In addition, aspects of the present invention provide methods and systems that have the capability to search, filter and compare accessed data for all of the participating hospitals using multiple criteria.

Other features and advantages in accordance with aspects of the present invention will become apparent from the following detailed description considered in connection with the accompanying drawings, which disclose multiple variations of the present invention. It should be understood, however, that the drawings are designed for the purpose of illustration only and not as a definition of the limits of the invention. Additional advantages and novel features in accordance with aspects of the present invention will also become more apparent to those skilled in the art upon examination of the following or upon learning by practice thereof.

### BRIEF DESCRIPTION OF THE DRAWINGS

In the drawings:

FIGS. 1A-1P present exemplary Graphic User Interface (GUI) screens, in accordance with aspects of the present invention;

FIG. 2 presents an example flow diagram of functions performed for online inventory and cost management, in accordance with aspects of the present invention;

FIG. 3 presents an exemplary system diagram of various hardware components and other features, in accordance with aspects of the present invention; and

FIG. 4 shows various features of an example networked computer system, including various hardware components and other features for use in conjunction with aspects of the present invention.

### DETAILED DESCRIPTION

Aspects of the invention will now be described in conjunction with the appended figures.

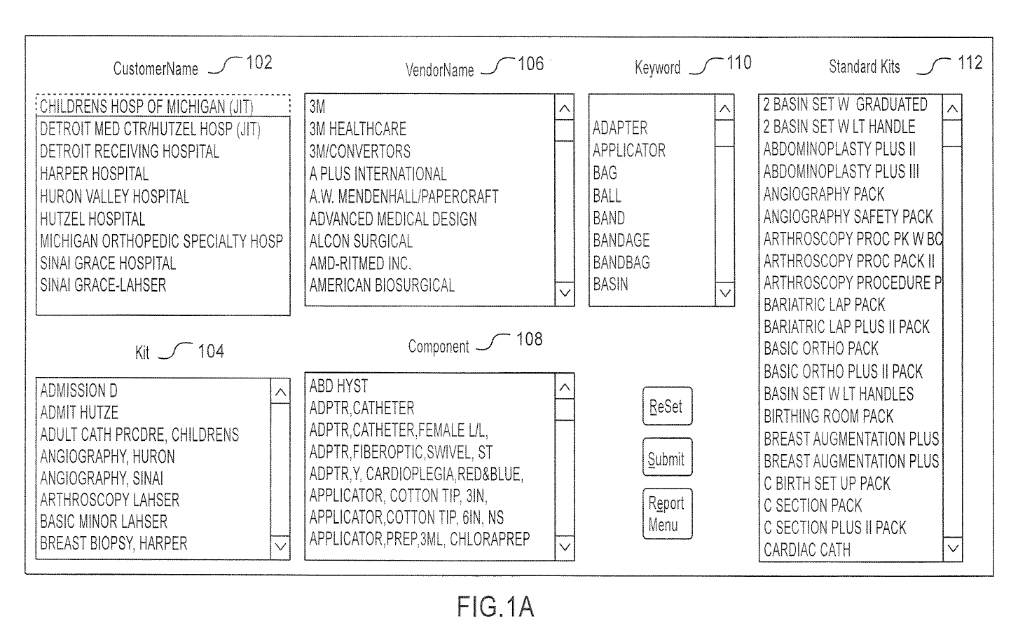

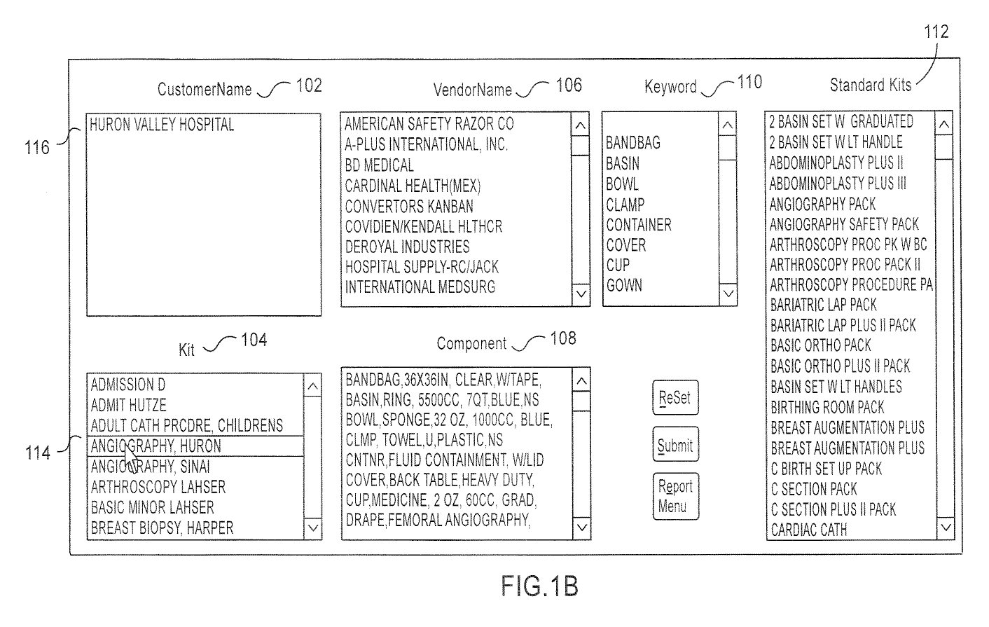

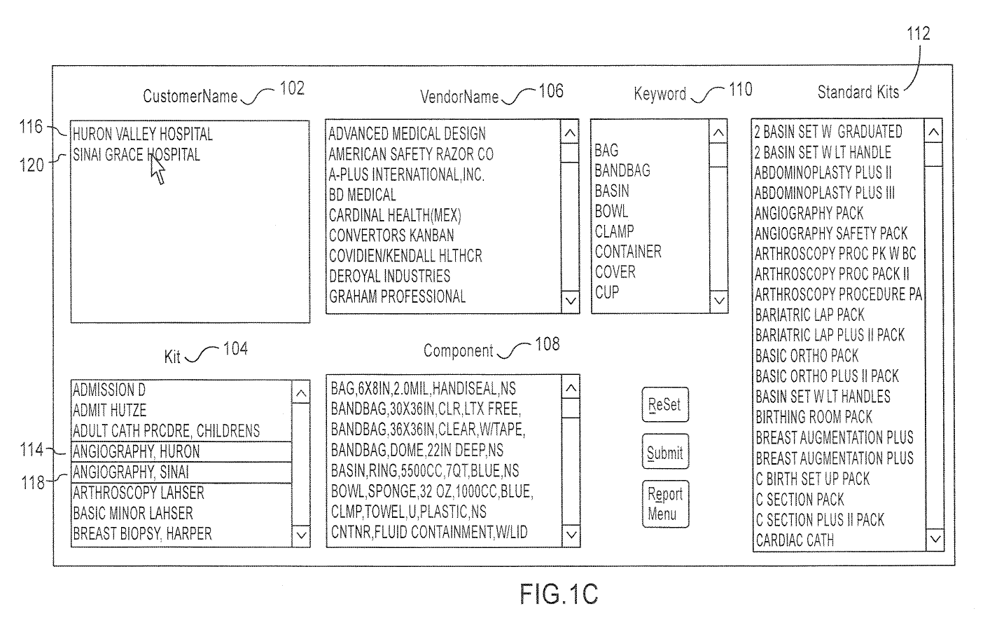

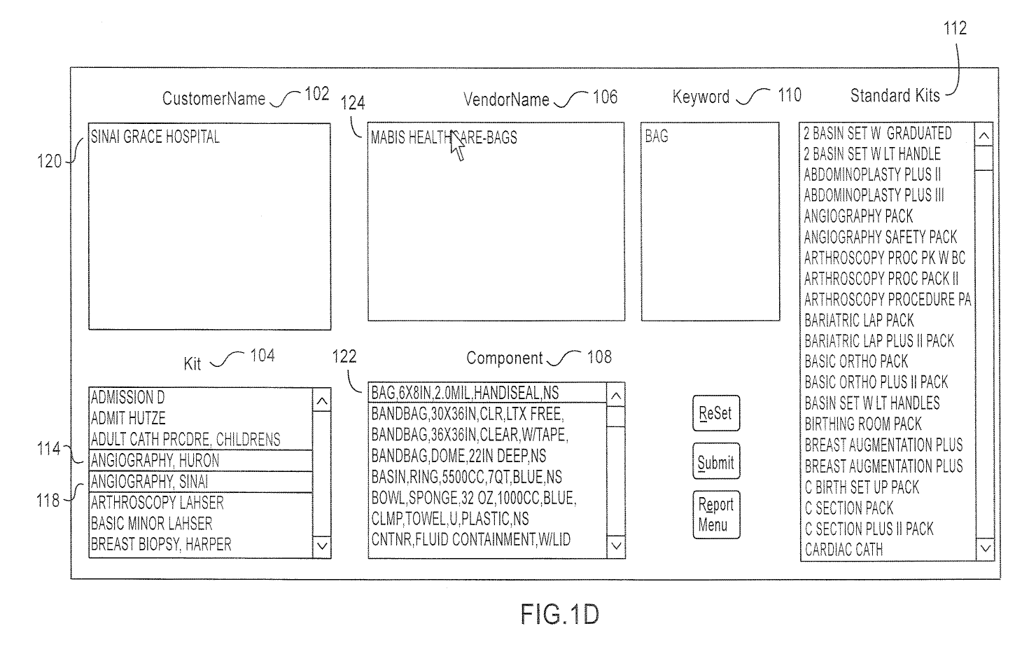

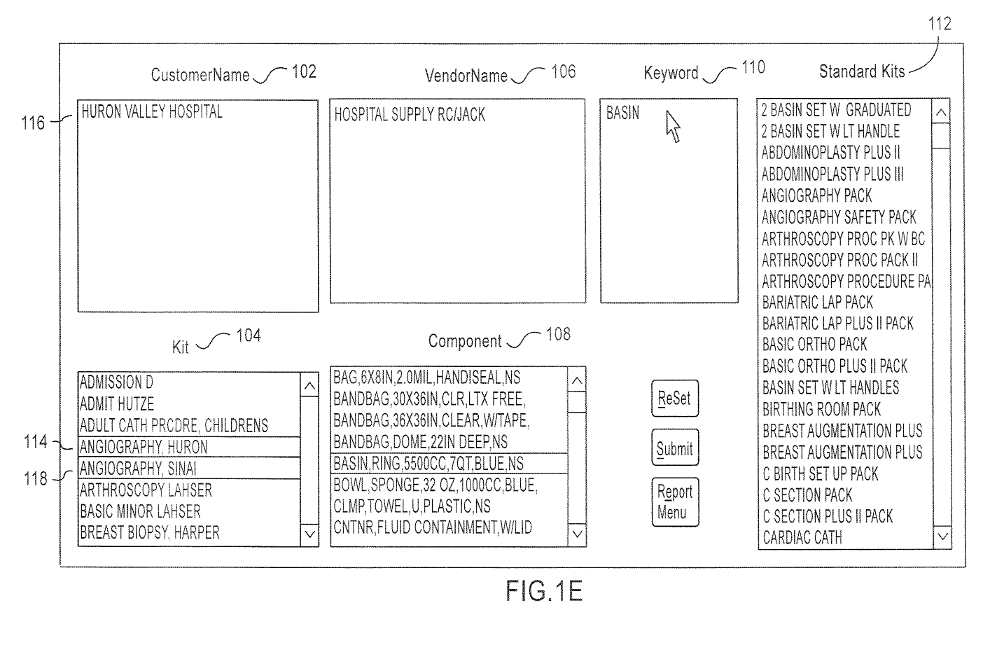

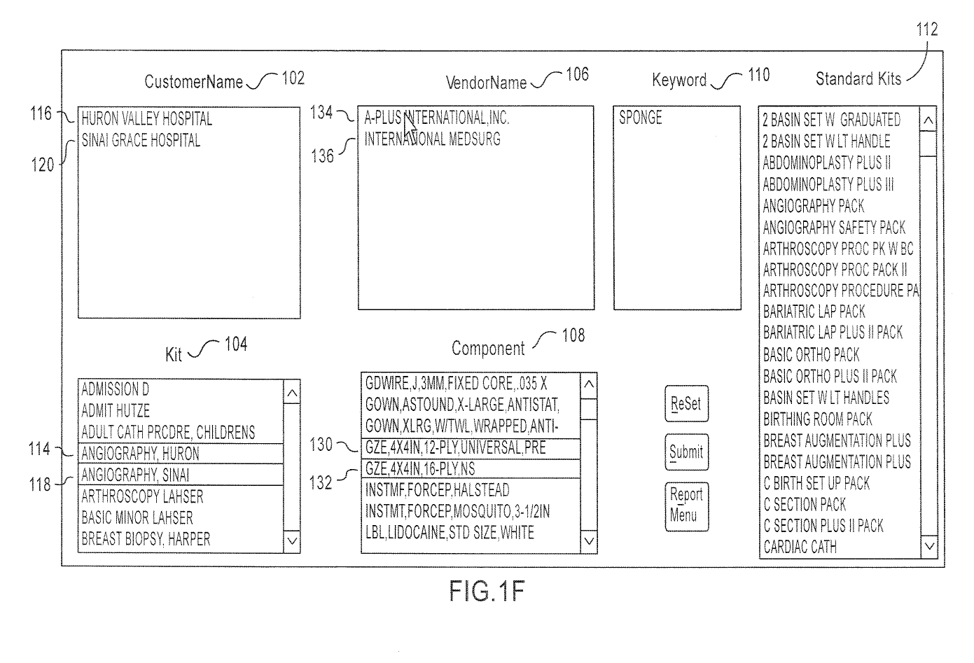

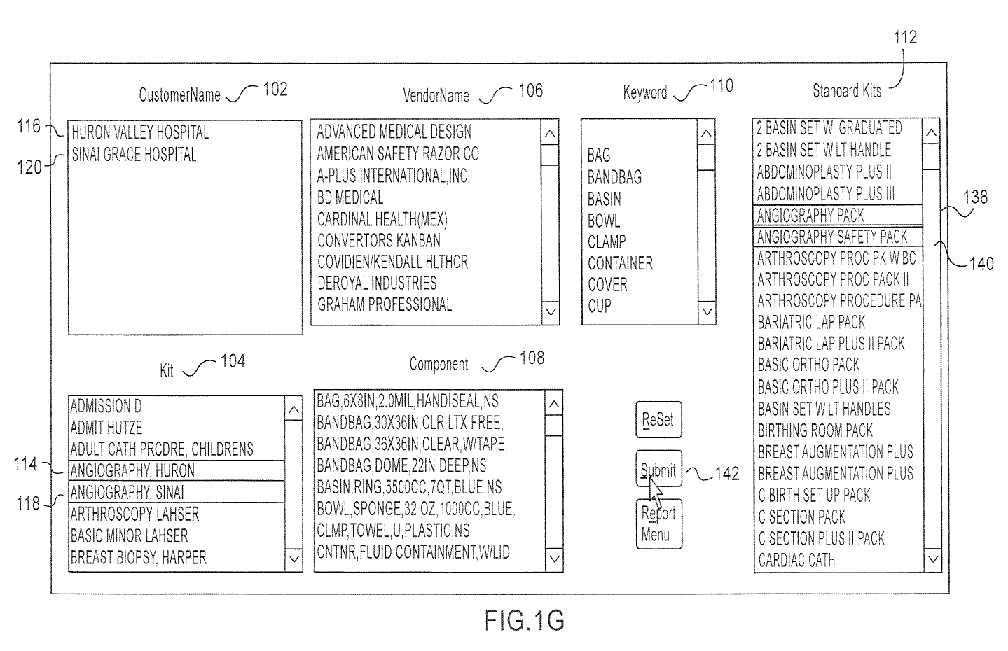

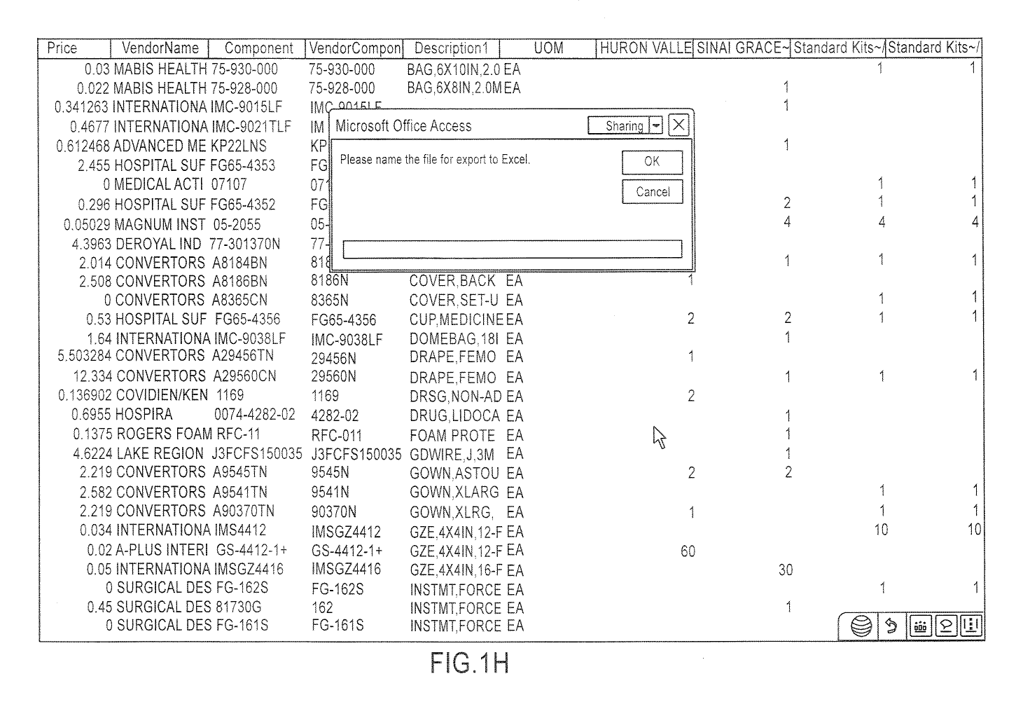

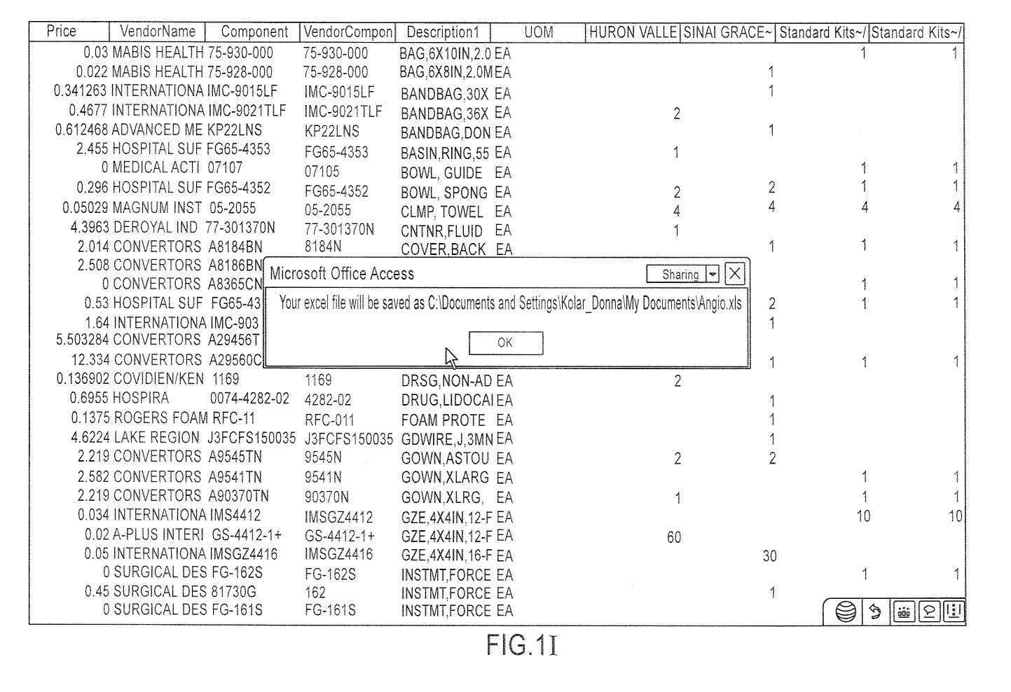

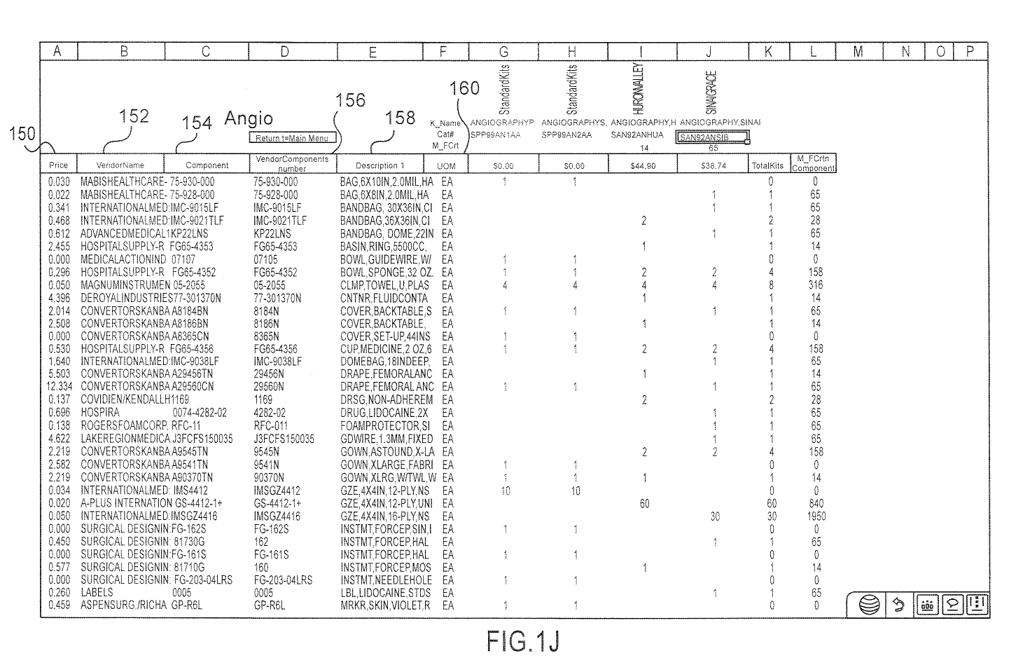

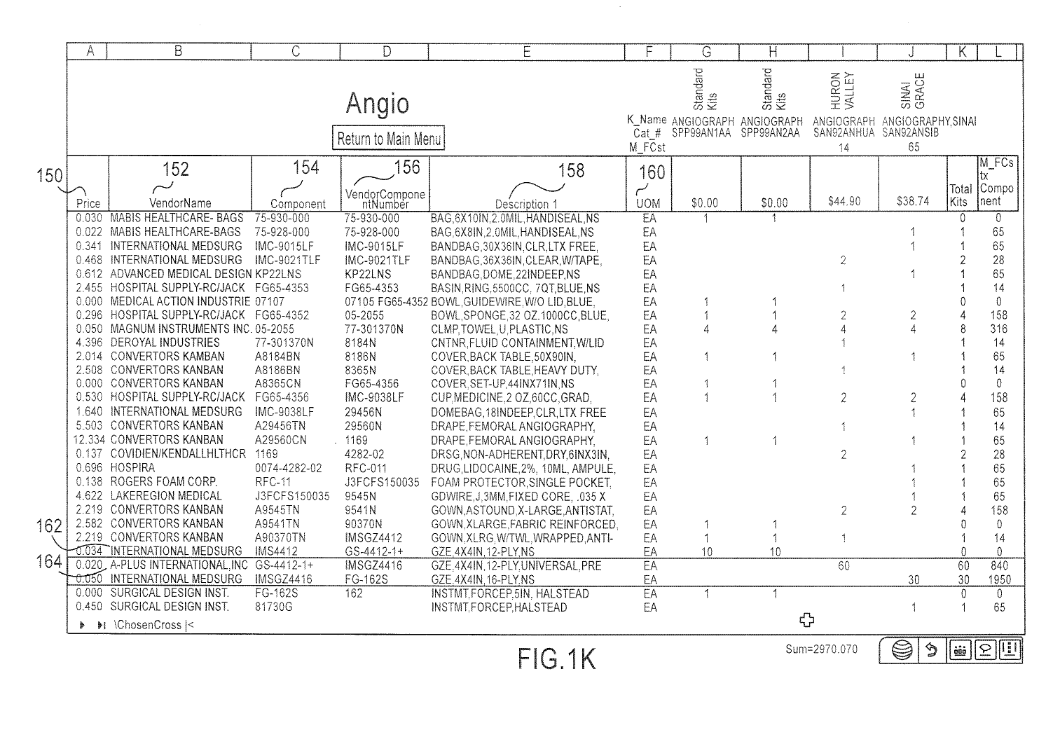

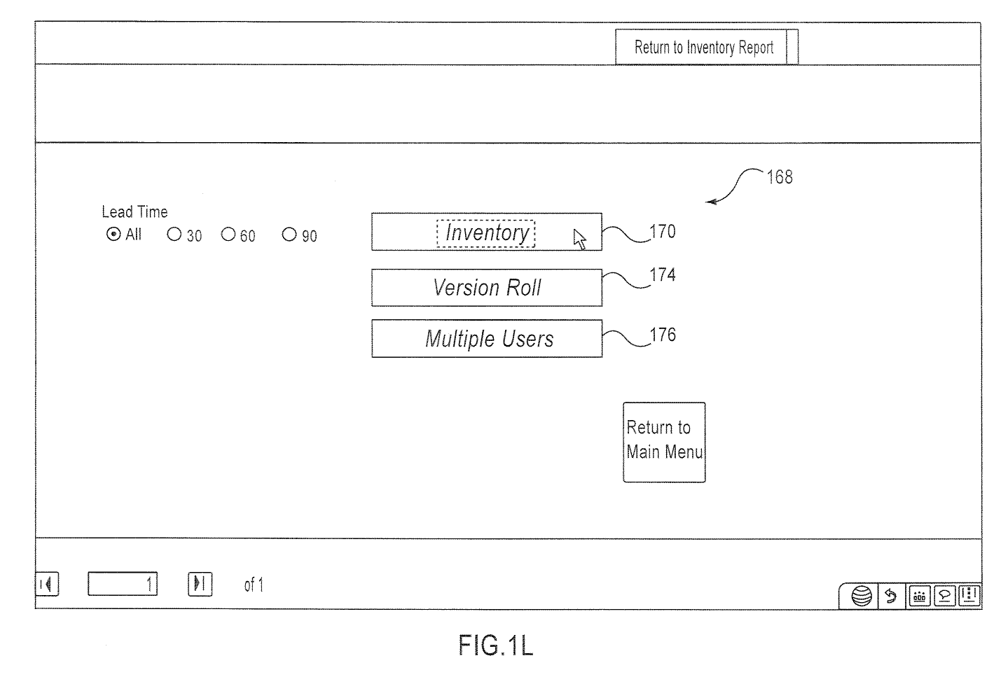

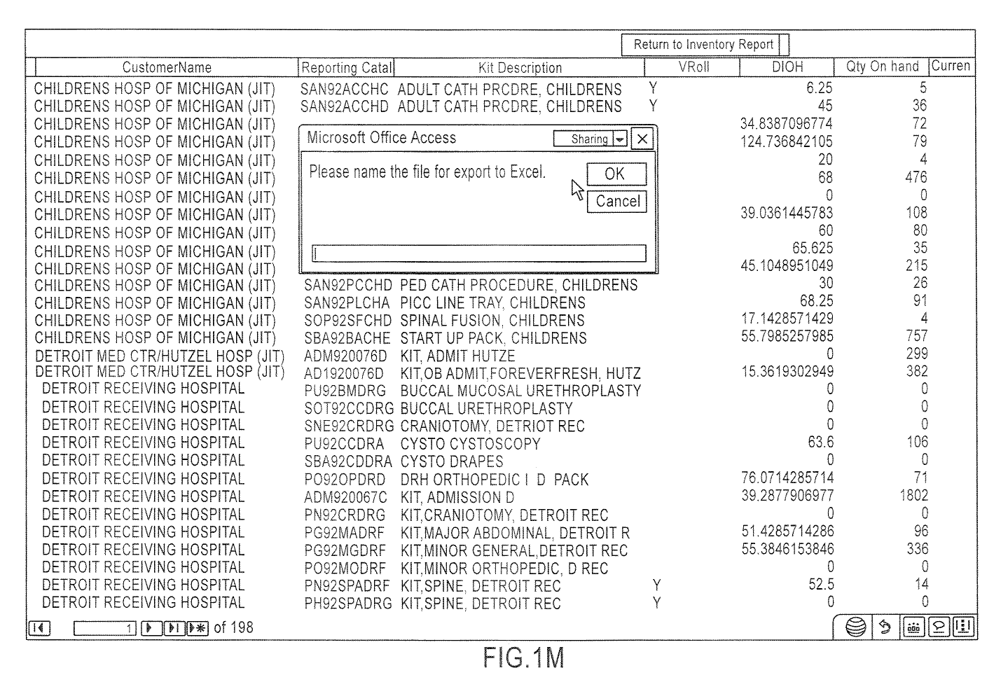

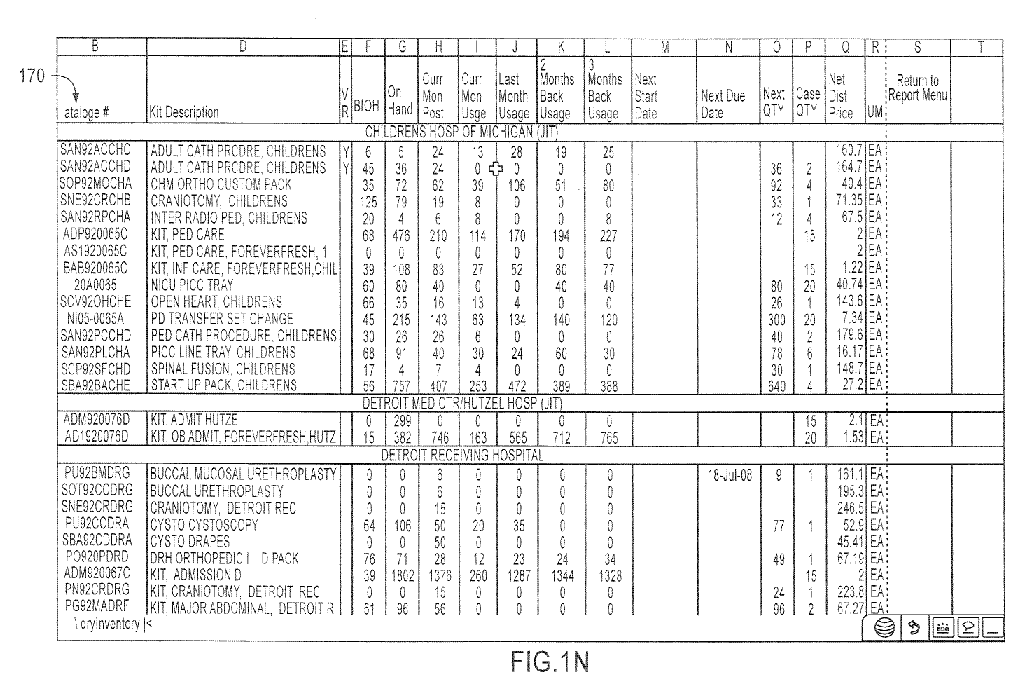

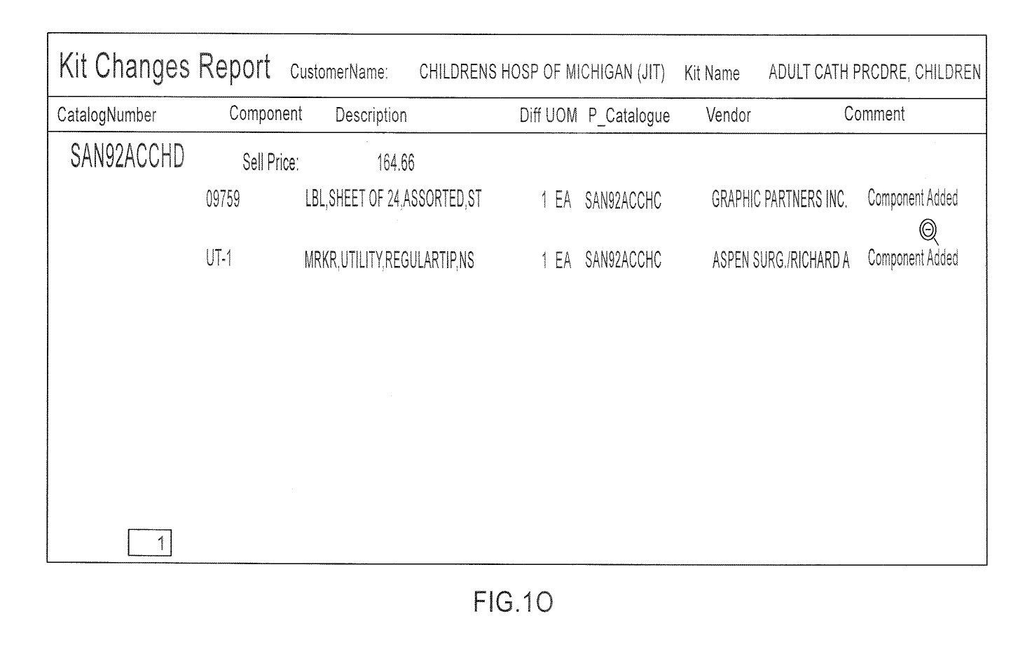

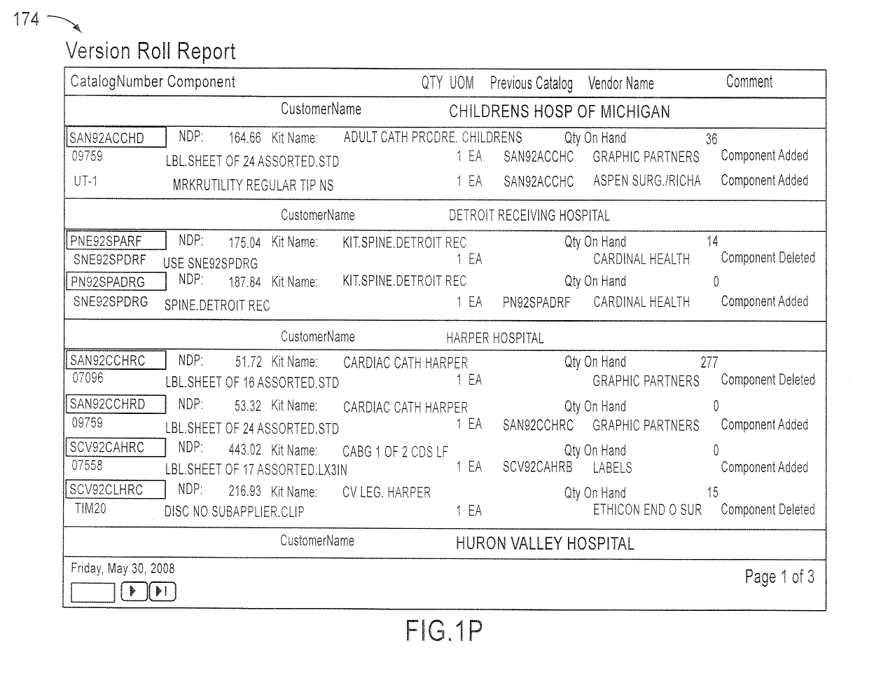

Referring to FIG. 1A, therein shown is an exemplary GUI screen in accordance with aspects of the present invention. The GUI screen shown in FIG. 1A may include a variety of filter or search boxes, each providing a drop-down list, pop-up menu, or other selection feature for selecting one or more customer names 102, one or more standard and/or custom kits 104, 112 used by the customers (e.g., hospitals), one or more components 108 within each kit 104, 112, one or more vendors 106 that provide the components 108 within the kits 104, 112, and/or one or more keywords 110 or other indicators designating a component of a kit 104, 112.

For example, as shown in FIG. 1B, if an analysis of angiography kits is desired, upon selection of the angiography kit 114 for Huron Valley Hospital 116, the components of the specific angiography kit 114 used at Huron Valley Hospital 116 appear in the Component box 108, and the vendors associated with the components appear in the VendorName box 106. In an aspect, the components of a specific kit may have a flag indicating materials that patients may have an increased sensitivity to within the component. For example, a component of a specific kit may have a flag with an “L” indicating the component contains latex.

In accordance with aspects of the present invention, multiple selections may be made across multiple boxes. For example, as shown in FIG. 1C, a selection of the Sinai angiography kit 118 may be made, along with the selection of the Huron angiography kit 114. In this example, the Component box 108 will show all the components used in the two kits, and the VendorName box 106 will display all the vendors associated with the components of the two kits 114, 118.

Additionally, if an analysis of a specific component used in the Huron and Sinai angiography kits 114, 118 is desired, a selection of the component can be made. For example, as shown in FIG. 1D, upon making a selection of a BAG, 6×8IN component 122 in the Component box 108, the VendorName box 106 displays the name of the vendor of this particular component, Mabis Healthcare 124, and the CustomerName box 102 displays the name of the hospital using the specific component in its angiography kits, Sinai Grace Hospital 120. Similarly, as shown in FIG. 1E, upon making a selection of a BASIN, RING, 5500CC component 126 in the Component box 108, the VendorName box 106 displays the name of the vendor of this particular component, Hospital Supply-RC/JACK 128, and the CustomerName box 102 displays the name of the hospital using the specific component in its angiography kits, Huron Valley Hospital 116.

In accordance with aspects of the invention, if an analysis of two components is desired simultaneously, as shown in the exemplary GUI screen in FIG. 1F, upon selection of two different gauze components 130, 132, GZE, 4×4IN, 12-PLY and GZE, 4×4IN, 16-PLY, the respective manufacturers 134, 136 are displayed in the VendorName box 106, and the hospitals 116, 120 using these components in their kits are displayed in the CustomerName box 102.

In accordance with an aspect of the invention, standardization of the angiography kits used at Huron and Sinai hospitals may be desired for a variety of reasons, including cost savings. As shown in FIG. 1G, for example, the angiography kits 138, 140 in the Standard Kits box 112 may be selected for analysis. Upon clicking or otherwise selecting the “Submit” button 142, for example, the analysis of the standard angiography kits 138, 140 may be presented as a Microsoft Excel® report file or other similar spreadsheet or other data report file. In accordance with aspects of the present invention, the report may also be presented on a display and in print form, among other presentation formats. FIGS. 1H and 1I show the creation of an exemplary Microsoft Excel® report file for the selected standard angiography kits 138, 140. A sample Microsoft Excel® report file for the selected standard angiography kits is shown in FIGS. 1J and 1K (expanded view of 14

Referring to FIG. 1K, the sample report may include information,

such as: price for each component 150; vendor name 152; component number 154; vendor component number 156 (if any); description of the component 158; unit of measure 160; and a comparison of the two standard angiography kits (shown in columns G and H) with the Huron and Sinai angiography kits (respectively shown in columns I and J). As shown in FIG. 1K, each of the standard kits (shown in columns G and H) includes 10 units of gauze, each at a price of $0.034 from vendor International Medsurge 162, while the Huron Valley kit (shown in column I) includes 60 units of gauze, each at a price of $0.020, from vendor A-Plus International, Inc. 164, and the Sinai kit (shown in column J) includes 30 units of gauze, each at a price of $0.050. In this example, column K shows the total number of components in a kit, and column L shows the monthly forecast multiplied by the number of components used. Based on this information, decisions may be made whether to standardize the custom kits by changing, for example, vendor and component unit quantity, and whether standardization would be cost effective. In an aspect, an automated savings potential calculation may be performed calculating the potential costs savings for standardizing the custom kits.

Similar reports may be produced based on any selected items in the filter boxes shown in FIG. 1A.

In accordance with aspects of the present invention, FIG. 1L shows an exemplary GUI screen showing a report menu 168, including an Inventory Report 170 (e.g., with lead time 30, 60 or 90 days, or all inventory), a Version Roll Report 174, and a Multiple Users Report 176. In accordance with aspects of the present invention, the Version Roll Report 174, an example of which is shown in FIG. 1P, provides a comparison of a current version of a custom kit with previous versions of the kit, if any. The Multiple User Report 176 shows whether a specific kit is used in multiple locations within the participating hospitals.

FIG. 1M shows the creation of an exemplary Inventory Report 170, which may be presented as a Microsoft Excel® report file or other similar spreadsheet report file. In accordance with aspects of the invention, the Inventory Report may also be presented on a display and in print form, among other presentation formats.

Referring to FIG. 1N, therein shown is a sample Inventory report, in accordance with aspects of the present invention. The report may be organized by participating hospitals/healthcare facilities, and may show the various kits used by each participating hospital. The report may show, for example, the catalog number (i.e., the kit number) (column B), the kit description (column D) and whether or not a particular kit has been version rolled (column E). In addition, there may be an indicator of available kit quantities (e.g., whether there are more or less available kits when compared to a predetermined number). Further, the Inventory Report may show, among other data, the days of inventory on hand (DIOH, column F), the actual on hand inventory (column G), the current monthly forecast (column H), the current monthly usage (column I), last month's usage (column J), usage for two or more months back (columns K and L), the next start date the kit will be going into production (column M), the next due date the kit is expected to be ready to be delivered to the warehouse (column N), the next quantity that will be produced (column O), how many units per case (column P), the net distributor price (column Q), and unit of measure (column R).

Based on this information, in accordance with aspects of the present invention, a specific hospital may desire to make a modification in one of the kits it uses. For example, for the CHM ORTHO Custom Pack used at Children's Hospital of Michigan shown in FIG. 1N, there are 72 kits actually on hand, and the next production date is known. Therefore, the hospital may plan how and when to implement a modification in this kit. In an aspect, automated usage alerts may be generated by the system altering the hospital of the usage of a particular kit. For example, the system may generate an alert if the hospital's usage of the kit is forecasted to be plus or minus thirty percent of the monthly planned usage or the previous month's usage. The hospital may use this alert to plan how and when to implement a modification in this kit.

In accordance with aspects of the invention, a hospital or other user may also desire to compare two different versions of kit, for example. Taking as an example the ADULT CATH Procedure kits used at Children's Hospital of Michigan shown in FIG. 1N, the catalog numbers (SAN92ACCHC and SAN92ACCHD) and the indicator in column E show that version C has been rolled into version D. Upon selecting version D, in accordance with aspects of the present invention, a Kit Changes Report is produced, as shown in FIG. 1O. The Kit Changes report may show what has been added and/or removed from the preceding version (C), as compared with the current version (D). In the example shown in FIG. 1O, a label and a marker have been added to the preceding version.

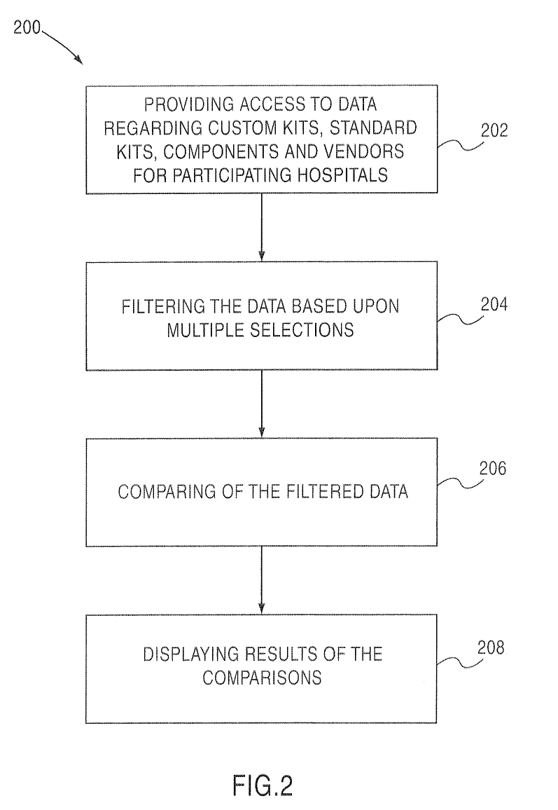

Referring now to FIG. 2, therein shown is an example flow diagram 200 of functions performed for online inventory and cost management in accordance with aspects of the present invention. In one variation, in step 202, access is provided to data regarding custom kits, standard kits, kit components within each kit, and vendor data for each component for a variety of participating hospitals. The data may be refreshed with updated customer-specific information. In one aspect, the data may be refreshed weekly with updated customer-specific information. In another aspect, the data may be refreshed in real-time (e.g., as the users modify the components within a kit, the data is continually refreshed with updated customer-specific information).

In step 204, upon selection of multiple parameters of the custom kits, standard kits, kit components within each kit, and vendor data for each component, the accessed data is filtered accordingly. Next, in step 206, in one variation, comparisons, such as quality, quantity, and cost comparisons, may be provided for the filtered data, and at step 208, the comparison results may be displayed to the user. In an aspect, the user may create custom reporting displaying the comparison results for the filtered data. For example, the user may create inventory reports, version roll reports, multiple user reports, reports for auditing vendors for contract compliance, kit changes reports, and reports comparing custom kits with standard kits, among other customized reports.

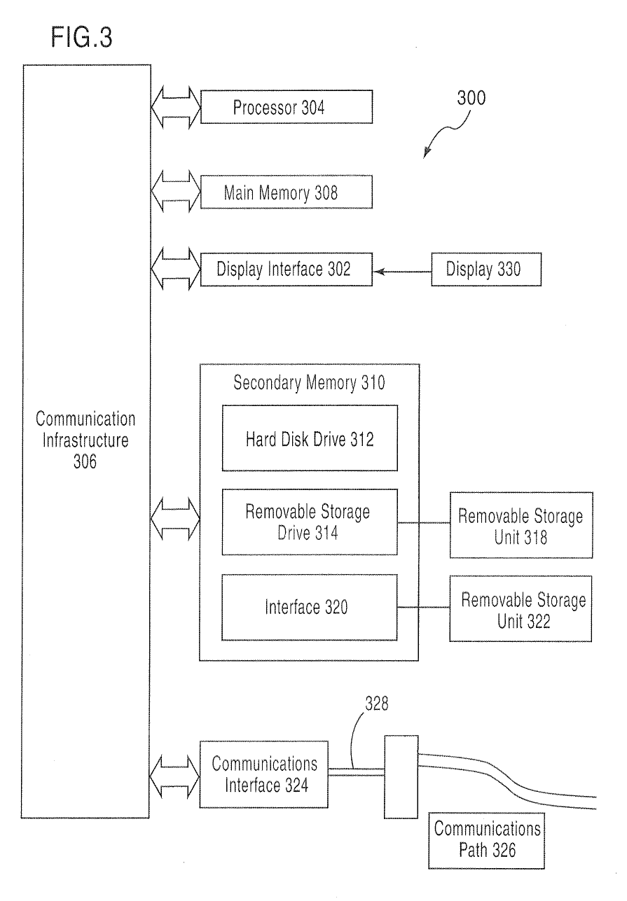

Variations of the present invention may be implemented using hardware, software, or a combination thereof and may be implemented in one or more computer systems or other processing systems. In one variation, aspects of the present invention are directed toward one or more computer systems capable of carrying out the functionality described herein. An example of such a computer system 300 is shown in FIG. 3.

Computer system 300 includes one or more processors, such as processor 304. The processor 304 is connected to a communication infrastructure 306 (e.g., a communications bus, cross-over bar, or network). Various software variations are described in terms of this exemplary computer system. After reading this description, it will become apparent to a person skilled in the relevant art(s) how to implement aspects of the present invention using other computer systems and/or architectures.

Computer system 300 can include a display interface 302 that forwards graphics, text, and other data from the communication infrastructure 306 (or from a frame buffer not shown) for display on a display unit 330. Computer system 300 also includes a main memory 308, preferably random access memory (RAM), and may also include a secondary memory 310. The secondary memory 310 may include, for example, a hard disk drive 312 and/or a removable storage drive 314, representing a floppy disk drive, a magnetic tape drive, an optical disk drive, etc. The removable storage drive 314 reads from and/or writes to a removable storage unit 318 in a well-known manner. Removable storage unit 318, represents a floppy disk, magnetic tape, optical disk, etc., which is read by and written to removable storage drive 314. As will be appreciated, the removable storage unit 318 includes a computer usable storage medium having stored therein computer software and/or data.

In alternative variations, secondary memory 310 may include other similar devices for allowing computer programs or other instructions to be loaded into computer system 300. Such devices may include, for example, a removable storage unit 322 and an interface 320. Examples of such may include a program cartridge and cartridge interface (such as that found in video game devices), a removable memory chip (such as an erasable programmable read only memory (EPROM), or programmable read only memory (PROM)) and associated socket, and other removable storage units 322 and interfaces 320, which allow software and data to be transferred from the removable storage unit 322 to computer system 300.

Computer system 300 may also include a communications interface 324. Communications interface 324 allows software and data to be transferred between computer system 300 and external devices. Examples of communications interface 324 may include a modem, a network interface (such as an Ethernet card), a communications port, a Personal Computer Memory Card International Association (PCMCIA) slot and card, etc. Software and data transferred via communications interface 324 are in the form of signals 328, which may be electronic, electromagnetic, optical or other signals capable of being received by communications interface 324. These signals 328 are provided to communications interface 324 via a communications path (e.g., channel) 326. This path 326 carries signals 328 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 380, a hard disk installed in hard disk drive 370, and signals 328. These computer program products provide software to the computer system 300. Aspects of the present invention are directed to such computer program products.

Computer programs (also referred to as computer control logic) are stored in main memory 308 and/or secondary memory 310. Computer programs may also be received via communications interface 324. Such computer programs, when executed, enable the computer system 300 to perform the features of various aspects of the present invention, as discussed herein. In particular, the computer programs, when executed, enable the processor 310 to perform such features. Accordingly, such computer programs represent controllers of the computer system 300.

In one variation of the present invention, which is implemented using software, the software may be stored in a computer program product and loaded into computer system 300 using removable storage drive 314, hard drive 312, or communications interface 320. The control logic (software), when executed by the processor 304, causes the processor 304 to perform the functions of the invention as described herein. Another variation is implemented primarily in hardware using, for example, hardware components, such as application specific integrated circuits (ASICs). Implementation of the hardware state machine so as to perform the functions described herein will be apparent to persons skilled in the relevant art(s).

In yet another variation, aspects of the present invention are implemented using a combination of both hardware and software.

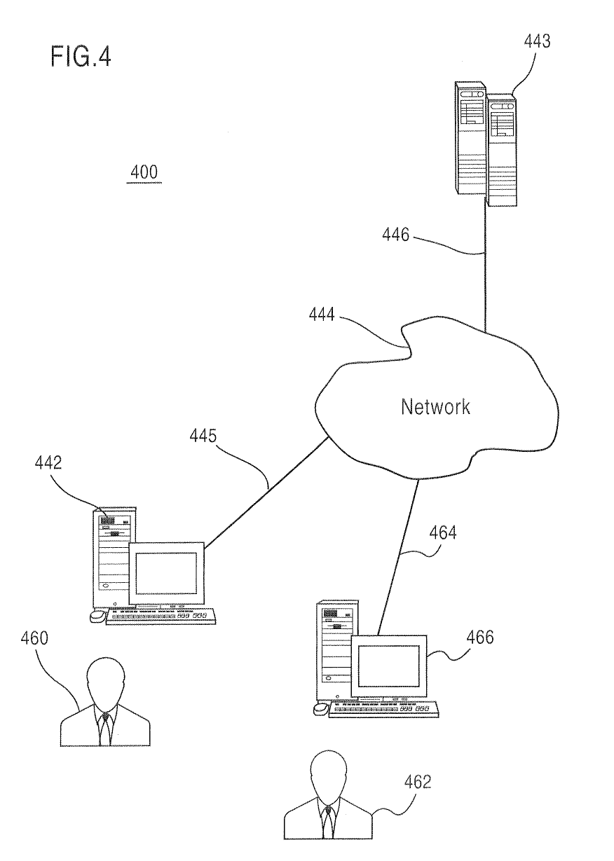

FIG. 4 shows a communication system 400 usable in accordance with aspects of the present invention. The communication system 400 includes one or more users 460, 462 (also referred to interchangeably herein as one or more “accessors”) and one or more mobile devices 442, 466. In one variation, data for use in accordance with aspects of the present invention is, for example, downloaded and/or accessed by users 460, 464 via terminals 442, 466, such as laptop computers or wireless devices, including PDAs or other hand-held wireless devices, coupled to a server 443, such as a laptop computer, mobile telephone or other device having a processor and a repository for data and/or connection to a repository for data, via, for example, a network 444, such as the Internet or an intranet, and couplings 445, 446, 464. The couplings 445, 446, 464 may include, for example, wired, wireless, or fiberoptic links. In another variation, the method and system operate in a stand-alone environment, such as on a single mobile device.

While aspects of the present invention have been described in connection with various features, it will be understood by those skilled in the art that variations and modifications of such aspects described above may be made without departing from the scope thereof. Other aspects will be apparent to those skilled in the art from a consideration of the foregoing or from a practice thereof.

1. A method for procedure pack inventory management implemented on a suitably programmed computer comprising a processor and a display, the method comprising:
accessing data including healthcare facility data, custom and standard procedure pack data, component data for each procedure pack, and vendor data;
filtering, via the processor, the accessed data based upon at least one selected from a group consisting of the healthcare facility data, the custom and standard procedure pack data, the component data for each procedure pack, and the vendor data;
comparing results of the filtering; and
displaying results of the comparison via the display.

2. The method of claim 1, further comprising:
selecting modifications to the component data for a procedure pack based upon comparing the results of the filtering.

3. The method of claim 2, wherein the modifications to the component data for the procedure pack are based upon one selected from the group consisting of cost savings for the procedure pack, available components for the procedure pack, an amount of inventory on hand, a forecast of usage of the procedure pack, a date the procedure pack will go into production, and the net vendor price for the procedure pack.

4. The method of claim 3, wherein the modifications include standardizing the component data for the procedure pack.

5. The method of claim 2, further comprising:
creating a new version of the procedure pack based upon the modifications to the component data for the procedure pack,
wherein the component data for the procedure pack includes a tag identifying the current version of the procedure pack.

6. The method of claim 5, further comprising:
comparing the components of the new version of the procedure pack with a previous version of the procedure pack,
wherein the comparison identifies the differences between the two versions of the procedure pack.

7. The method of claim 1, wherein displaying the results of the comparison further comprises:
generating a report,
wherein the report is at least one selected from a group consisting of an inventory report, a version roll report and a multiple users report.

8. The method of claim 1, further comprising:
associating at least one keyword with the accessed data,
wherein the keyword designates a specific component within the procedure pack; and
filtering the accessed data based upon the keyword.

9. A system for procedure pack inventory management, the system comprising:
means for accessing data including healthcare facility data, custom and standard procedure pack data, component data for each procedure pack, and vendor data;
means for filtering the accessed data based upon at least one selected from a group consisting of the healthcare facility data, the custom and standard procedure pack data, the component data for each procedure pack, and the vendor data;
means for comparing results of the filtering; and
means for displaying results of the comparison.

10. A system for procedure pack inventory management, the system comprising:
a data repository;
a user interface for accessing data stored in the data repository including healthcare facility data, custom and standard procedure pack data, component data for each procedure pack, and vendor data;
a filtering module for filtering the accessed data based upon at least one selected from a group consisting of the healthcare facility data, the custom and standard procedure pack data, the component data for each procedure pack, and the vendor data;
a comparing module for comparing results of the filtering; and
a display for displaying results of the comparison via the user interface.

11. The system of claim 10, further comprising:
a selecting module for selecting modifications to the component data for a procedure pack based upon comparing the results of the filtering.

12. The system of claim 11, wherein the modifications to the component data for the procedure pack are based upon one selected from the group consisting of cost savings for the procedure pack, available components for the procedure pack, an amount of inventory on hand, a forecast of usage of the procedure pack, a date the procedure pack will go into production, and the net vendor price for the procedure pack.

13. The system of claim 12, wherein the modifications include standardizing the component data for the procedure pack.

14. The system of claim 11, further comprising:
a creating module for creating a new version of the procedure pack based upon the modifications to the component data for the procedure pack,
wherein the component data for the procedure pack includes a tag identifying the current version of the procedure pack.

15. The system of claim 14, further comprising:
the comparing module for comparing the components of the new version of the procedure pack with a previous version of the procedure pack,
wherein the comparison identifies the differences between the two versions of the procedure pack.

16. The system of claim 10, further comprising:
an associating module for associating at least one keyword with the accessed data,
wherein the keyword designates a specific component within the procedure pack; and
the filtering module for filtering the accessed data based upon the keyword.

17. A system for procedure pack inventory management, the system comprising:
a processor;
a user interface functioning via the processor; and
a repository accessible by the processor;
wherein accessing data includes healthcare facility data, custom and standard procedure pack data, component data for each procedure pack, and vendor data is provided;
wherein the accessed data is filtered based upon at least one selected from a group consisting of the healthcare facility data, the custom and standard procedure pack data, the component data for each procedure pack, and the vendor data;
wherein results of the filtering are compared; and
wherein results of the comparison are displayed.

18. A computer program product comprising a computer usable medium having control logic stored therein for causing a computer to perform procedure pack inventory management, the control logic comprising:
first computer readable program code means for accessing data including healthcare facility data, custom and standard procedure pack data, component data for each procedure pack, and vendor data;
second computer readable program code means for filtering the accessed data based upon at least one selected from a group consisting of the healthcare facility data, the custom and standard procedure pack data, the component data for each procedure pack, and the vendor data;
third computer readable program code means for comparing results of the filtering; and
fourth computer readable program code means for displaying results of the comparison.
